(19) United States
(12) Patent Application Publication
(10) Pub. No.: US 2023/0126325 A1
(43) Pub. Date: Apr. 27, 2023

(54) TEMPORARY ANATOMY ILLUMINATION

(71) Applicant: GYRUS ACMI, INC. D/B/A OLYMPUS SURGICAL TECHNOLOGIES AMERICA, Westborough, MA (US)

(72) Inventors: Jordan C. Avalos, Littleton, MA (US); Kurt G. Shelton, Bedford, MA (US); Bo Yang, Westborough, MA (US); Sergey A. Bukesov, Acton, MA (US)

(21) Appl. No.: 18/048,733

(22) Filed: Oct. 21, 2022

Related U.S. Application Data
(60) Provisional application No. 63/262,932, filed on Oct. 22, 2021.

(51) Int. Cl. A61B 1/06; A61B 1/018; A61B 1/00

(52) U.S. Cl. CPC A61B 1/0676; A61B 1/018; A61B 1/0014; A61B 1/00085

(57) ABSTRACT

Various aspects of methods, systems, and use cases may be used to deploy a light source to a targeted organ location via a working channel of an endoscope, such as in a manner that leaves the working channel free for use by another instrument and that can avoid requiring an additional channel, beyond the working channel, for illumination of a target region.

### PRIORITY CLAIM

This application claims the benefit of priority to U.S. Provisional Patent Application No. 63/262,932, filed Oct. 22, 2021, the contents of which are hereby incorporated by reference.

### BACKGROUND

Medical endoscopes were first developed in the early 1800s and have been used to inspect inside the body. An endoscope may be used to facilitate delivery of laser energy from a laser to an organ or other internal target location. Some endoscopes may be used to treat a kidney stone with a laser.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an endoscope system with a laser or other energy source, an endoscope, and a working channel to temporarily illuminate a portion of patient anatomy.

FIG. 2A is a graph of the wavelength and intensity of Ca₃Ga₄O₉:Tb³⁺/Zn²⁺ phosphor at a variety of temperatures.

FIG. 2B is a graph of time and normalized wavelength of Ca₃Ga₄O₉:Tb³⁺/Zn²⁺ phosphor at a variety of temperatures.

### DETAILED DESCRIPTION

One factor in making and using endoscopes, including single-use endoscopes, is providing beneficial features while keeping the diameter as small as reasonably possible. Including various endoscope channels in an endoscope can consume space along the endoscope, thereby increasing its diameter. Examples of such channels can include a working channel, an illumination channel, an image sensing channel, an irrigation fluid channel, a suction channel, etc. An endoscope can include one or more light sources at a distal end of the endoscope. The systems and techniques described herein can help provide desired endoscope functionality while helping reduce or minimize the precious real estate used in the desirably thin diameter endoscope device. The apparatus and methods described herein can apply to medical, veterinary, and/or engineering devices that are used to view cavities using a scope; this may include all the different variations of endoscopes, laparoscopes, ureteroscopes, colonoscope, arthroscope, etc. that fall within the general term “scope” as understood by a person having ordinary skill in the art. For ease of description and understanding, examples presented herein will refer to an “endoscope” or “scope”, which terms can be used interchangeably.

The systems and techniques described herein can help conserve space along the endoscope, such as by making an illumination channel(s) unnecessary. For example, the endoscope may be 8-10 French. The systems and techniques described herein can relate to a temporary standalone light that may be placed in the body (e.g., a kidney), such as during the medical procedure, and removed thereafter.

One or more illumination light sources may be delivered to the patient anatomy (e.g., a kidney or other organ or other target location), such as to remain there throughout the procedure. At the end of the procedure, the light source(s) are then removed from the patient anatomy (e.g., a kidney or other organ or other target location).

The light source may be a single device or multiple devices from which light emits.

The light source(s) may be allowed to free float, or may be temporarily attached to the organ such as at the tissue wall, or attached to another device available in the area (e.g., a safety guidewire). In another embodiment, the light source may be configured to optionally attach or otherwise mate with the distal end of the endoscope.

The small light source(s) may be delivered independently of the endoscope (e.g., via a separate tool, or a guidewire, etc.), or may be delivered via the working channel of the endoscope, or via a device in the working channel of the endoscope (e.g., a stone basket).

Where the light source is very small, it can be retrieved after the procedure, such as through the working channel of the scope, such as with a retrieval basket device or other device configured to attach to, grasp, or otherwise capture the light source. Alternatively or additionally, the light source can be grabbed and “towed” from the end of the endoscope during its removal. Alternatively or additionally, if the light source(s) is small enough, it can be expelled naturally by the patient.

The light source(s) can be any shape suitable for the delivery method employed, e.g., very small circular/oval light source devices.

For example, the light source may be tubular (analogous to that of a neon tube) for delivery through the working channel of the scope, and may be constructed so as to collapse in on itself for pulling back through the working channel when the procedure is otherwise complete.

Another example may use a thin sleeve on the exterior of a distal portion of the scope, such as, when positioned in the kidney or other organ or other target location within the patient, the sleeve may be retracted to “bunch up” on itself in a ring around the distal portion of scope while being used for illumination.

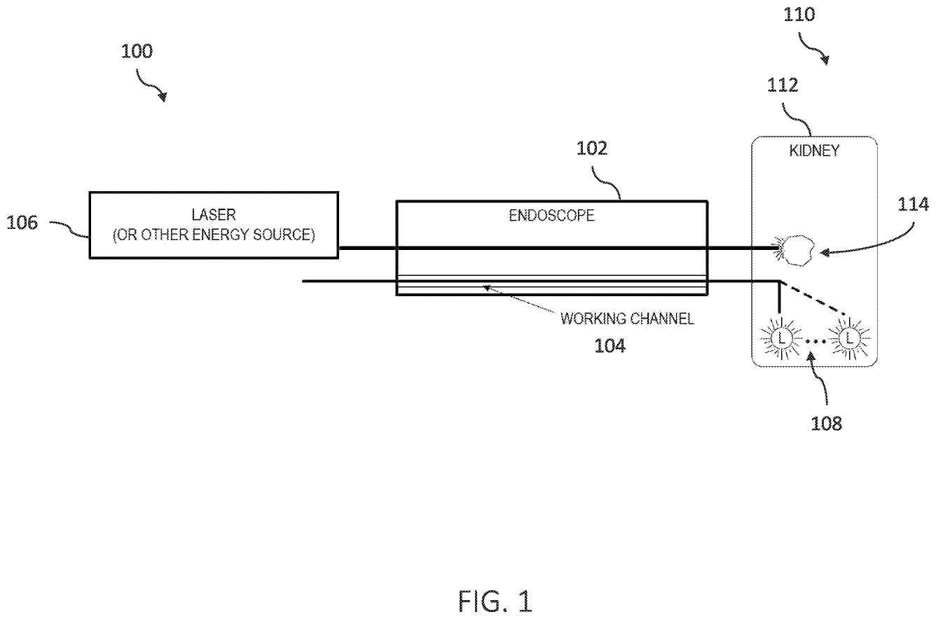

FIG. 1 illustrates an example of portions of an endoscope system 100. The endoscope system 100 can include an endoscope 102. The endoscope 102 can include or be coupled to a laser fiber or a laser or other energy source 106, such as for performing laser lithotripsy, or any other ancillary treatment device to be used at the target location within the patient. The endoscope 102 can include a working channel 104 such as for use to deliver an illumination light source to temporarily illuminate a portion of patient anatomy 110. The energy source 106 may pass through the working channel 104 to reduce the number of channels in the endoscope 102 and the diameter of the endoscope 102.

The endoscope 102 may optionally include an additional channel for the laser source 106, such as in addition to the working channel 104. However, the endoscope 102 need not include any additional channels. Specifically, the endoscope 102 need not include an illumination channel. The endoscope 102 may include a guidewire channel. Optionally, the laser source 106 may use the working channel 104 such as for communicating laser light via a laser fiber inserted via the working channel 104. The working channel 104 can extend from a proximal end to a distal end of the endoscope 102. Additionally or alternatively, the endoscope 102 may include a channel for suction and/or irrigation.

The laser source 106 can provide therapeutic energy to the working area 112 (e.g., a kidney or other targeted organ) to perform the desired medical function. For laser lithotripsy, the laser source 106 may include or be coupled to a laser fiber to be passed through the endoscope 102 toward one or more target kidney stones/calculi 114. Such lithotripsy can be used to fragment and/or dust the stones for ultimate removal from the body. A different energy source than a laser may additionally or alternatively be used, depending on the procedure, such as an ultrasound energy source, a radio frequency (RF) or other electromagnetic energy source, a plasma energy source, etc. Regardless of the particular treatment being performed internally to the patient under visual or imaging observation of the scope, it can be helpful to illuminate the target location such as to help the physician or other end-user better observe the target location through such imaging or visualization.

Accordingly, the endoscope system 102 can include one or more light sources 108. The one or more light sources 108 may be a single light source 108 or a plurality of light sources 108. The one or more light sources 108 may be omnidirectional. The one or more light sources 108 may be deployed into the working area 112 (e.g., a kidney or other targeted organ) for the duration of a procedure. This can leave the working channel 104 of the scope unoccupied by an illumination light source or conduit, such that one or more other instruments may be passed through the working channel 104 of the endoscope, when the one or more light sources are deployed at their desired location(s) outside of the working channel 104 of the endoscope, such as beyond the distal tip of the endoscope in or near the target region for performing the procedure. This can also help avoid the need for an illumination channel, running separate from and parallel to the working channel 104.

For example, a deployment device can be positioned through the working channel 104, to deliver or provide one or more light sources 108 to the working area 112 (e.g., a kidney or other targeted organ). The deployment device may disengage from the one or more light sources 108. After delivering and disengaging from the one or more light sources 108, the deployment device may then be removed from the working channel 104 during a later portion of the medical procedure. The one or more light sources 108 may be retrieved from the working area 112 (e.g., a kidney or other targeted organ) after the procedure, such as using the deployment device or a different retrieval device. For example, the one or more light sources 108 may be deployed so as to be permitted to free float in the working area 112 (e.g., a kidney or other targeted organ). For example, the one or more light sources 108 may be delivered to the working area 112 (e.g., a kidney or other targeted organ) and detached from the deployment device extending through the working channel 104. For example, the one or more light sources 108 can be sized to pass through the working channel 104. The one or more light sources 108 may travel to the working area 112 (e.g., a kidney or other targeted organ) at a distal end of the endoscope 102, such as without requiring deployment via the working channel 104. For example, the one or more light sources 108 may be sized with a dimension larger than a diameter of the working channel 104 and equal to or smaller than a diameter of the distal end of the endoscope 102. The one or more light sources 108 may be configured to be permitted to remain tethered in the working area 112 (e.g., a kidney or other targeted organ), such as to the deployment device, a guidewire, or a distal end of the endoscope 102. Optionally, the one or more light sources 108 may be configured with an attachment mechanism, such as to permit the one or more light sources 108 to be attached to a wall of the working area 112 (e.g., a kidney or other targeted organ).

For example, the one or more light sources 108 may include a light emitting diode (LED). The one or more light sources 108 may include a battery and be battery powered. The one or more light sources 108 may be sized, shaped, or otherwise configured pass through the patient naturally, such as via a ureter. Alternatively, the one or more light sources 108 may be configured to be retrieved from the working area 112 (e.g., a kidney or other targeted organ). For example, the one or more light sources 108 may be retrieved using the deployment device. Alternatively or additionally, the one or more light sources 108 may be retrieved using a basket, such that the basket can grab, capture, or attach to the one or more light sources 108. The one or more light sources 108 can then be removed through the working channel 104. In an example, the one or more light sources 108 can be removed by grabbing or attaching the one or more light sources 108, such as using a basket or a deployment device, and removing the one or more light sources 108 by removing the endoscope 102. The one or more light sources 108 can include a magnet or an electromagnet, and a distal end of the endoscope 102 can include a magnet or an electromagnet. Thus, the one or more light sources 108 can attach to the distal end of the endoscope 108 when the magnets are in proximity. Or, the one or more light sources 108 can attach to the distal end of the endoscope 108 when the electromagnets are activated by delivering a small current.

The one or more light source 108 can be tubular in shape. For example, the one or more light sources 108 can create a light sleeve extending around an exterior portion of the endoscope 102. A height of the one or more light sources 108 in the light sleeve can be substantially negligible compared to an overall diameter of the endoscope 102, such that the light sleeve does not substantially add to the diameter of the endoscope 102. The one or more light sources 108 of the light sleeve can be connected to a deployment device extending through the working channel 104. The deployment device can be retracted, while still attached to the one or more light sources 108 of the light sleeve. The retraction of the deployment device may cause the one or more light sources 108 of the light sleeve to bunch, such as at a distal end of the endoscope 102. For example, the one or more light sources 108 of the light sleeve can bunch to create a ring or ball of the one or more light sources 108. The deployment device can be retracted further, such as to cause the one or more light sources 108 of the light sleeve to be pulled inside the working channel 104. The one or more light sources 108 can then be pulled through the working channel 104 to remove the one or more light sources 108 from the working area 112 (e.g., a kidney or other targeted organ). Optionally, the one or more light sources 108 of the light sleeve can be detached from the deployment device before or during the procedure and optionally reattached to the deployment device such as for positioning or retrieval.

The one or more light sources 108 can include an illuminated guidewire. The illuminated guidewire can be passed through the working channel 104 or through a guidewire channel of the endoscope 102. The illuminated guidewire can include an illuminated surgical fiber. The illuminated guidewire can be illuminated using visible or infrared light. The endoscope 102 can include one or more fluorescent portions such as on an exterior distal end of the endoscope 102. The fluorescent material can forms one or more rings around the endoscope 102. The illuminated guidewire can cause illumination of the fluorescent portions of the endoscope 102, which, in turn, can help provide illumination toward a nearby target region.

In some embodiments, the one or more light sources 108 can originate external to the patient. For example, the one or more light sources 108 may be positioned external to the endoscope 102 at a proximal end of the endoscope 102 that is exterior to the patient. The one or more light sources 108 may travel through the working channel 104 and then exit the working channel (e.g., tethered or untethered) such as to illuminate the working area 112 (e.g., a kidney or other targeted organ). In an example, the working channel 104 can remain open at both ends.

The one or more light sources 108 can include a fluorescent fluid that can be configured to be delivered into the working area 112 (e.g., a kidney or other targeted organ). For example, the fluorescent fluid can be injected into the working area 112 (e.g., a kidney or other targeted organ) such as via the working channel 104. The fluorescent fluid can be expelled by passing through the patient naturally. The fluorescent fluid can illuminate the working area 112 (e.g., a kidney or other targeted organ). Under illumination, a portion of the working area 112 (e.g., a kidney or other targeted organ) can be imaged. For example, the imaging can include ultrasound, infrared, or other imaging technique (from visible light to invisible light). The fluorescent fluid can aid in differentiating between soft tissue and/or the calculi stone 114.

In imaging, the imaging device can acquire one or more images of the working area or the working area 112 (e.g., a kidney or other targeted area), e.g., without fluorescent fluid. For example, the imaging device can include an ultrasound device, an infrared device, sonar, or other imaging device. The imaging device may include image processing capabilities, or can be coupled to an image-processing device with such capabilities. For example, the image processing may distinguish between soft tissue of the working area 112 (e.g., a kidney or other targeted organ) and the calculi stone 114. Or, the imaging processing can distinguish between areas of different temperature, such that the imaging device can distinguish between soft tissue of the working area 112 (e.g., a kidney or other targeted organ) and the calculi stone 114. In some embodiments, the imaging process, by distinguishing between the soft tissue of the working area 112 (e.g., a kidney or other targeted organ) and the calculi stone 114 can indicate the tissue and the calculi stone 114 on the image. Multiple imaging probes or devices may be used to create an image that allows the user to “see behind” the calculi stone 114 (e.g., one imaging method or device captures the calculi stone 114 and another imaging method or device captures an image behind the calculi stone 114).

The one or more light sources 108 may include one or more long persistence phosphorescent materials. Long-persistence luminescence, or afterglow, is a phenomenon in which the material shows long-lasting luminescence after the cessation of an excitation source. The excitation source can include ultraviolet (UV) or visible (VIS) light. For example, the long persistence phosphorescent material can emit light for hours after the excitation source is removed or turned off. One or more portions of an exterior of the endoscope 102 can include or be coated with the long persistence phosphorescent material. For example, Ca₃Ga₄O₉:Tb³⁺/Zn²⁺ phosphor may be used as a component of the long persistence phosphorescent material. Ca₃Ga₄O₉:Tb³⁺/Zn²⁺ phosphor has luminescence appear under visible light excitation in the wavelength region of 400-600 nm.

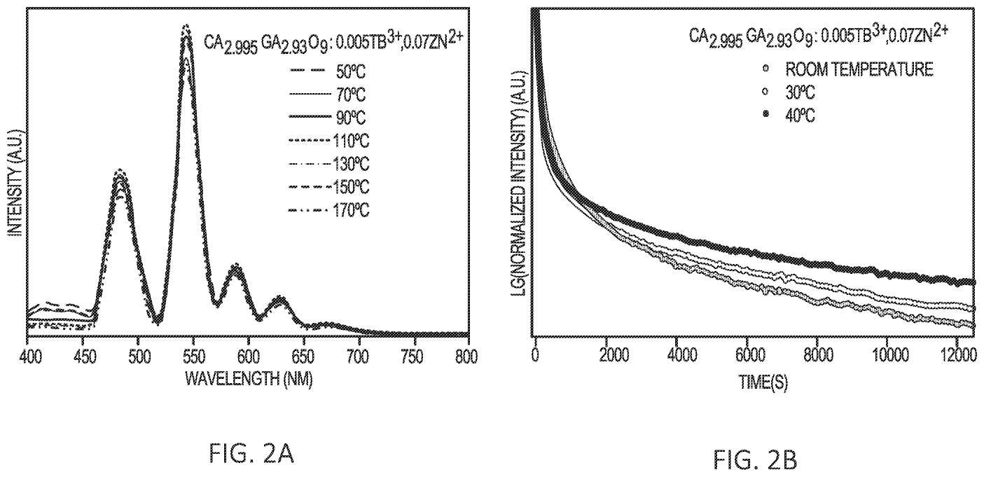

FIG. 2A illustrates a graph of the wavelength and intensity of Ca₃Ga₄O₉:Tb³⁺/Zn²⁺ phosphor at a variety of temperatures.

FIG. 2B illustrates a graph of time and normalized wavelength of Ca₃Ga₄O₉:Tb³⁺/Zn²⁺ phosphor at a variety of temperatures. Accordingly, the properties of Ca₃Ga₄O₉:Tb³⁺/Zn²⁺ phosphor are suited for illuminating the working area 112 (e.g., a kidney or other targeted organ).

The system 100 can include a near infrared light source external to the patient. The endoscope 102 can include an infrared detector. The near infrared window (e.g., an optical window or therapeutic window) can define the range of wavelengths from 650 to 1350 nm. Light may have its maximum depth of penetration in tissue deep structures at the near infrared window.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

What is claimed is:

1. An endoscope comprising:
a working channel portion defining a channel within the endoscope, the channel configured to receive one or more light sources;
wherein the one or more light sources are delivered to an organ via the channel.

2. The endoscope of claim 1, wherein the one or more light sources includes an illumination guidewire.

3. The endoscope of claim 2, wherein an exterior of the endoscope comprises one or more fluorescent materials such that the illumination guidewire illuminates the one or more fluorescent materials.

4. The endoscope of claim 1, wherein the one or more light sources include a light source located outside of the endoscope channel and outside of a body, wherein a light generated by the one or more light sources travels through the working channel of the endoscope to the organ.

5. The endoscope of claim 1, wherein the one or more light sources are configured to free float in the organ or affix to a tissue of the organ.

6. The endoscope of claim 5, wherein the one or more light sources are configured to be retrieved via a basket configured to grab the one or more light sources.

7. The endoscope of claim 5, wherein the one or more light sources include a magnet or electromagnet, and a distal end of the endoscope include an endoscope magnet or endoscope electromagnet.

8. The endoscope of claim 1, wherein the one or more light sources comprise a fluorescent fluid injected into the organ via the working channel.

9. A method comprising:
deploying, via an endoscope, one or more light sources into an organ of a patient;
leaving the one or more light sources in the organ during a procedure.

10. The method of claim 9, further comprising removing the one or more light sources after the procedure has been performed in the organ.

11. The method of claim 9, wherein leaving the one or more light sources in the organ during the procedure includes permitting the one or more light sources to free float in the organ during the procedure.

12. The method of claim 9, further comprising deploying the one or more light sources through a working channel of the endoscope using a deployment device that disengages from the one or more light sources.

13. The method of claim 12, further comprising, before disengaging the deployment device from the one or more light sources, attaching the one or more light sources to a wall of the organ.

14. The method of claim 12, further comprising, before disengaging the deployment device from the one or more light sources, attaching the one or more light sources to a distal end of the endoscope.

15. The method of claim 10, wherein removing the one or more light sources includes using a basket device to attach to the one or more light sources.

16. The method of claim 10, wherein removing the one or more light sources includes attaching the one or more light sources to the endoscope and further comprising removing the one or more light sources by removing the endoscope.

17. A method comprising:
deploying, through a working channel of an endoscope, one or more light sources into an organ of a patient using a deployment device;
disengaging the deployment device to leave the one or more light sources in the organ;
removing the deployment device from the working channel;
retrieving the one or more light sources after a procedure has been performed in the organ.
